(19) United States
(12) Patent Application Publication
(10) Pub. No.: US 2024/0008711 A1
(43) Pub. Date: Jan. 11, 2024

(54) HYGIENIC FOOTWEAR EQUIPMENT RACK UNIT

(71) Applicant: Mukesh Sharma, Surrey (CA)

(72) Inventor: Mukesh Sharma, Surrey (CA)

(21) Appl. No.: 17/648,542

(22) PCT Filed: Jun. 2, 2021
(86) PCT No.: PCT/IB2021/054822

§ 371 Date: Dec. 8, 2022

(51) Int. Cl. A47L 23/20; D06F 58/10; A43B 3/26; A61L 2/10; A61L 2/18

(52) U.S. Cl. CPC A47L 23/20; D06F 58/10; A43B 3/26; A61L 2/10; A61L 2/18; A61L 2202/11; A61L 2202/14; A47B 61/04

(57) ABSTRACT

A hygienic footwear equipment rack unit to maintain a hygienic environment of a premise and to prevent outdoor footwear handling. Slippers for indoor use are provided in various sizes. The slippers may be provided via a conveyor hanger with magnets to secure the slippers. Further provided may be a method of sanitizing outdoor shoes using UV lamps and hot air fans.

### CROSS-REFERENCE TO RELATED APPLICATIONS

This nonprovisional application claims priority based upon the following prior Patent Cooperation Treaty (PCT) application No. PCT/162021/054822 entitled: “Hygienic Footwear Equipment Rack Unit”, Jun. 2, 2021, in the name of Mukesh Sharma, which is hereby incorporated by reference for all purposes.

### FIELD OF THE INVENTION

The present specification relates generally to a storage rack for footwear, and more particularly to a hygienic storage rack for sanitization of footwear and provision of sanitized footwear.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

The invisible threat posed by outdoor environmental germs, whether viral, bacterial and/or fungal may be transmitted, transferred or carried unknowingly from outdoor footwear contaminating and/or cross-contaminating an indoor environment. Proper practices for handling of outdoor footwear are often not observed due to a lack of proper equipment or lack of user-friendly components. Thus, there is a need for a footwear handling system that promotes good hygienic practices as well as providing for handling of unsterilized outdoor footwear in a hands free and user friendly manner.

Therefore, there is a need to provide a system to protect an indoor clean and hygienic environment from contamination by outdoor footwear. Ideally, such a system should provide not only for the sanitization of outdoor footwear, but also for the provision of replacement footwear to be worn while within the indoor area. Such provided footwear is preferably adjustable to accommodate different foot sizes without compromising the space requirements of the system.

Accordingly, there remains a need for improvements in the art.

### SUMMARY OF THE INVENTION

In accordance with an aspect of the invention, there is provided a hygienic storage rack for sanitization of outdoor footwear and provision of sanitized footwear for use indoors.

According to an embodiment of the invention, there is provided [NTD: to be completed from claims once approved]

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

Other aspects and features according to the present application will become apparent to those ordinarily skilled in the art upon review of the following description of embodiments of the invention in conjunction with the accompanying figures.

### BRIEF DESCRIPTION OF THE DRAWINGS

Reference will now be made to the accompanying drawings which show, by way of example only, embodiments of the invention, and how they may be carried into effect, and in which:

FIG. 1 is a front perspective view of a hygienic footwear equipment rack according to an embodiment;

FIG. 2A is a exploded side perspective view of a slipper according to an embodiment;

FIG. 2B is a top view of an alternate embodiment of a slipper;

FIG. 3 is front view of the slippers mounted on a conveyor mechanism;

FIG. 4 is a perspective view of a pulley-type conveyor mechanism;

FIG. 5 is a perspective view of vertical chain conveyor mechanism;

FIG. 6 is a front view of the hygienic footwear equipment rack and conveyor mechanism; and

FIG. 7 is a side view of FIG. 6.

Like reference numerals indicated like or corresponding elements in the drawings.

### DETAILED DESCRIPTION OF THE INVENTION

The present invention relates generally to a storage rack for footwear, and more particularly to a hygienic storage rack for sanitization of footwear and provision of sanitized footwear.

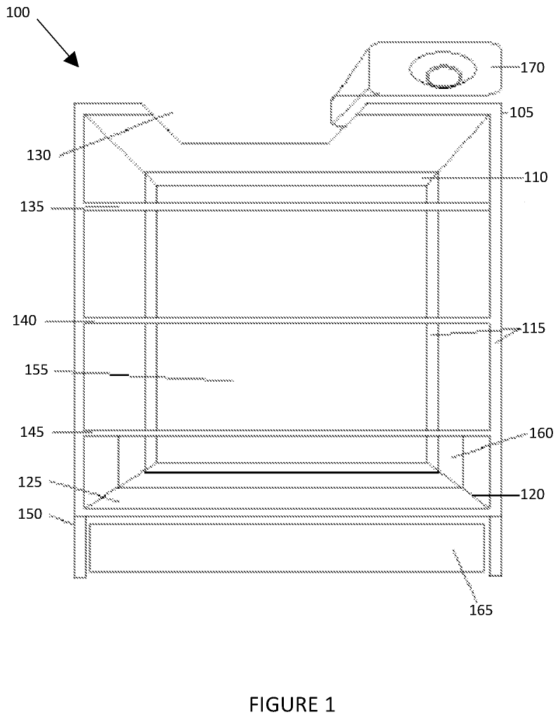

According to an embodiment as shown in FIG. 1, a hygienic footwear equipment rack 100 is assembled on a frame of metal or rigid plastic or similar material. The overall height, width and depth will be based on the desired capacity specifications for the equipment rack 100.

The frame is formed from a front 105, back 110, four sides 115 which extend as the legs 150 of the equipment rack 100, and a bottom 120. The bottom 120 is covered with a bottom plate 125 which allows for protective concealment of mechanical and electrical components as discussed below. The legs 150 provide for space (e.g. a few inches) below the bottom for other purposes and may further terminate in lockable wheels to allow for movement of the rack 100 if needed.

The top 130 of the frame includes an opening to permit the passage of hot air, UV light and other sterilizing elements into the equipment rack 100. A first shelf 135 is an open shelf used to hold unsterilized footwear for drying via hot air from a fixed or detachable hose coupled to a hot air blower fan 170 or another source. A second shelf 140 is a closed shelf used to hold footwear for exposure to UV light for sterilization. A third shelf 145 is used to hold sterilized slippers which are accessible through opening 155 between second shelf 140 and third shelf 145.

A partition 160 may be used to separate the lowest section between the third shelf 14 and bottom plate 125 into front and back halves.

165 is two detachable drawers set on the bottom of the frame, preferably horizontally adjacent and of similar size and shape. The back drawer is a sanitizing tray drawer containing a sanitizing liquid for sanitizing the bottom surface (sole) of footwear by having a user stand on or in the drawer. A raised pattern (e.g. crosshatching) may be provided on the surface to prevent slippage and allow circulation of liquid.

The front drawer 185 is similar in design and function, however, the surface is a carpet or similar material saturated with a sanitizing liquid to similarly enable the sanitizing of the footwear sole along with removal of dirt and debris through scrubbing on the surface. This drawer may also have a surface for wiping and/or drying the footwear sole. The drawers 165 may be manually actuated or driven by an electric motor (AC or DC), and may further include a remote device for activation.

A fan 170, preferably driving warmed or hot air, is provided on the top of the rack 100 to drive air through one or more fixed or detachable hoses 180 through the various shelves to assist in drying footwear and provide fresh air. A UV lamp may be provided on the hose outlets to further provide treatment for footwear contained in the rack 100. The end of the hose may be a split end to permit insertion into multiple shoes, with one or more UV lamps at each split end.

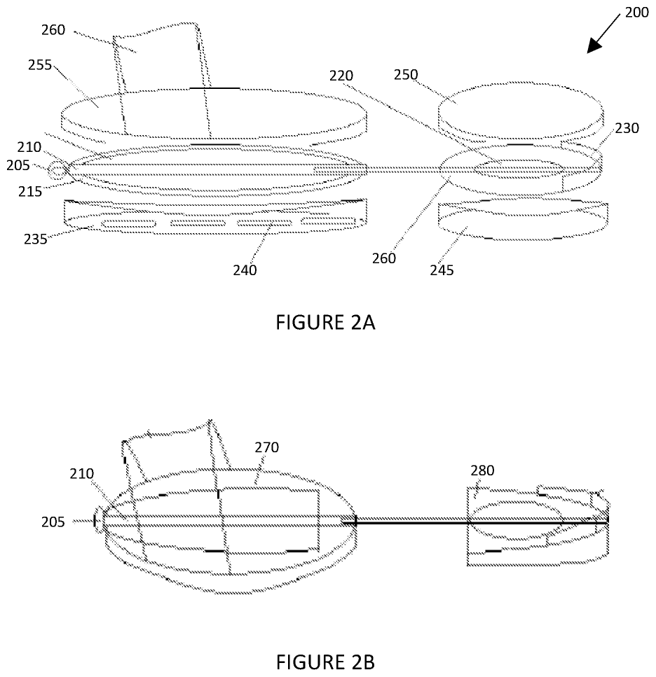

Slippers 200, as shown in FIGS. 2A and 2B, may be provided to be worn over shoes, socks or bare feet in place of footwear removed for sanitizing. The slippers 200 are adjustable to allow for fitting over different sizes of shoes and feet within a single design. The slippers 200 are intended for indoor use to avoid transmission of contaminants from outdoor shoes and to maintain the hygienic qualities of the indoor area.

A hanger 205 is provided at the front of the slipper 200 to allow the slipper 200 to be hung from a hook as discussed below. The main structure of the slipper 200 is provided by a central extendable telescopic-style bar 210 (metal or plastic) a toe support 215 and a heel support 220.

The toe support 215 is formed from a foot plate 225 with a bottom grip surface 235 which may include non-slip grooves 240, and a toe cushion 255 over the plate for comfort. A strap 260 of flexible fabric or rubber enables the user to slip on the slipper 200 and secure the slipper 200 to their foot with or without a shoe.

The heel support 220 is formed from a heel plate 265 with a rigid molded heel support 230 of metal or plastic and a bottom heel grip surface 245 and a heel cushion 250 over the heel plate 265.

Alternatively, as shown in FIG. 2B, the slippers 200 may be formed solely from rigid molded toe 270 and heel 280 elements, with an adjustable strap secured by elasticity or adjustable hook-and-loop fasteners to the foot.

Slippers 200 are therefore adjustable in length to accommodate different foot sizes. The slippers may be provided in a single adjustable size to accommodate, for example, US sizes 4 to 13. Alternatively, the slippers could be provided in separate sizes, such as small, medium and large, separate adjustable between a more limited size range e.g. US 3-7, 7-11 and 11-15.

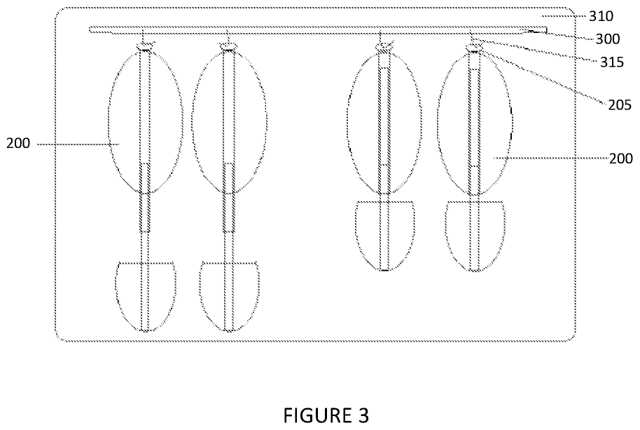

As shown in FIGS. 3, 4 and 5, a motorized conveyor system 300 may be provided to allow for rotation and presentation of the slippers 200 to the user. The motorized conveyor mechanism is a track 310 (either a chain or belt) driven by an axel and pulley system to present a vertical loop for the track.

On the track are hooks 315 for holding slippers 200. The hooks may be plastic or metal hooks, magnetic strips or hooks, or hook-and-loop patches designed to hold the slippers 200 in place during movement of the track while also permitting for ready removal of the slippers 200 by the user when needed.

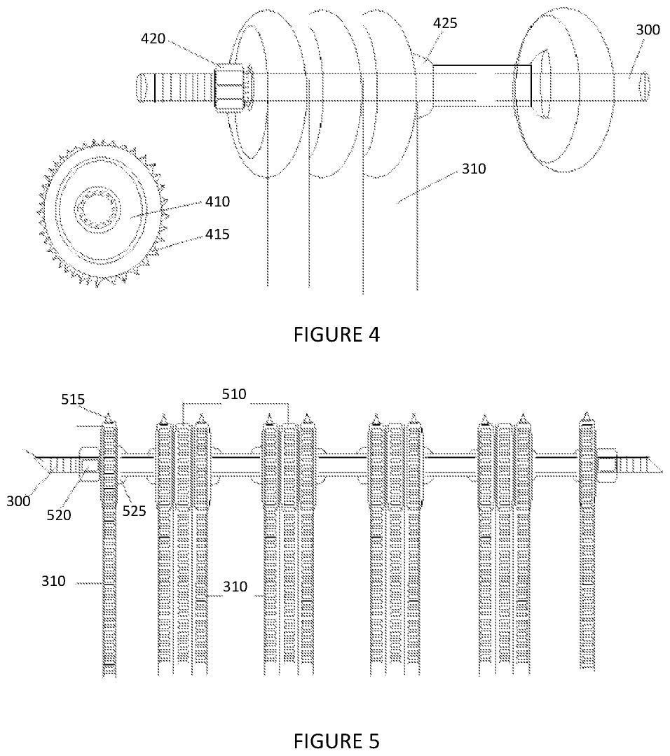

Referring to FIG. 4, the conveyor track 310 may be driven by an axel and free wheel mechanism, with a set of free wheels 410 with track grips 415 secured by a nut 420 and spacer 425 system to create a free moving conveyor system for track 310. Alternatively, as shown in FIG. 5, a series of sets of free wheels 510 and individual free wheels 515 secured through a series of nuts 520 and spacers 525.

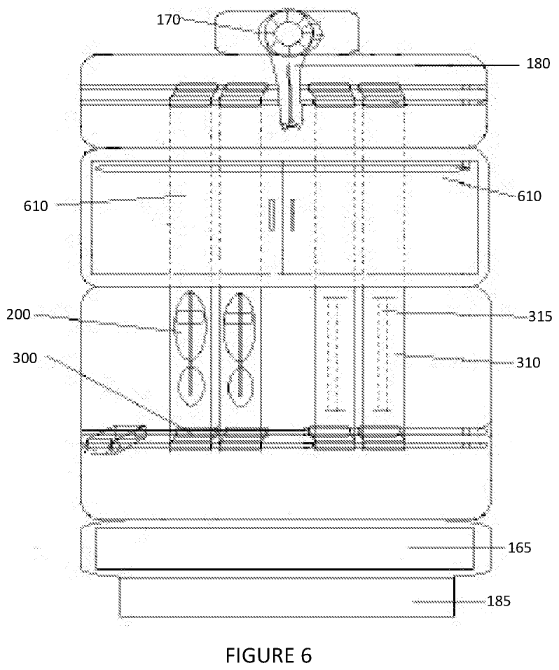

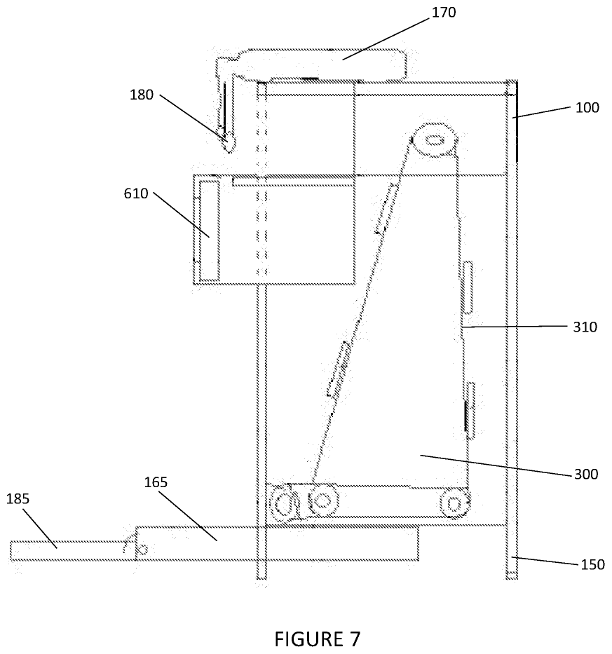

Referring to FIGS. 6 and 7, the motorized conveyor mechanism 300 allows for fresh sanitized slippers 200 to be moved to the front of the rack 100 for ready access by the user. In order to minimize contact, the motorized conveyor mechanism 300 may be operated by a foot paddle, a hand held remote control, a motion sensor or similar device.

Doors 610 may be provided to cover the shoe compartment to prevent airborne contamination. The doors may be constructed as single or double doors from any suitable material (glass, plastic, metal, wood), although preferably transparent or windowed to allow for a view of the slippers 200 inside the shoe compartment. The doors may also be power activated, similar to the motorized conveyor 300, to enable hands-free opening of the doors. Preferably, the doors may be automatically triggered to open when the motorized conveyor is activated.

The conveyed slippers 200 allow for the user to wear sanitized footwear before entering a clean and hygienic premise. Additionally, the sanitizing of the outdoor shoes may reduce the spread of environmental germs, viruses, bacterial and fungal infections which may otherwise be carried by the outdoor footwear to remote locations.

It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

The present invention may be embodied in other specific forms without departing from the spirit or essential characteristics thereof. Certain adaptations and modifications of the invention will be obvious to those skilled in the art. Therefore, the presently discussed embodiments are considered to be illustrative and not restrictive, the scope of the invention being indicated by the appended claims rather than the foregoing description and all changes which come within the meaning and range of equivalency of the claims are therefore intended to be embraced therein.

I claim:

1. That the claim is, hygienic footwear equipment rack unit may be uniquely arranged assembly unit of its kind for footwear handling equipment unit, interim of health safety, cleanliness, environment hygiene of place and of footwear's need.

2. That the claim is, hygienic footwear equipment rack unit may be categorized in nine equipment levels servicing unit for footwear's, including outdoor footwear's handling separately with advance multiple functions of processes.
1. Placement of hygienic footwear equipment rack unit at indoor entry point.
2. Sanitize footwear bottom part with sanitizer filled liquid tray on ground level of the equipment unit before entering the premises.
3. Wiping the footwear bottom sole after sanitizing with carpet tray doormat or rug and or damp wet soaking material.
4. Keeping outdoor footwear on equipment rack unit and or replace with wearable indoor slippers over footwear and shoes from the magnetic conveyer belts and or chains hands free.
5. Dry and sterilize outdoor shoes from inside, for odor, sweat and infection or allergy causing germs by hot air blower plus UV lamp on open rack 2^{nd} level in open air of equipment unit.
6. Sterilize any kind of footwear from all sides with UV lamp rays in the closet shoe compartment on 3^{rd} level of equipment unit.
7. The top 1^{st} level of the equipment may use for hot air blower, power switches, timer switches, light switch and other electric components function switch for operations.
8. The hygienic footwear equipment rack unit may be fully automated to use from the distance hands free accept, hand held remote device for equipment's electric component functions remotely.
9. The tray drawers on the bottom floor 5^{th} and 6^{th} level of hygienic footwear equipment rack unit may be auto functional with ac/dc power electric motor attach with tray drawers for open close eject function by switch or by hand held remote control device.

3. That the claim is, hygienic footwear equipment rack unit may be placed in indoor premises at entry point of access area to cutoff or keeping separate indoor environment from outdoor environment for hygiene and health safety reasons.

4. That the claim is, hygienic footwear equipment rack unit may be comprised with electric components such as hot air blower or ultra violet lamps, magnetic conveyer motor belts and metal chains might be installed within the equipment unit at any level of or order of user's convenience in equipment unit.

5. That the claim is, with basic arrangement of hard solid board of metal bar on with hanger hooks may be placed at entry point of premises to hang newly designed slippers sets for use over the shoes or footwear's slip in slippers flap and or replacement of footwear with slippers for hygienic reasons before entering in a clean and hygienic place. This may also with no mechanical arrangements required to maintain.

6. That the claim is, hygienic footwear equipment rack units conveyer motor belts or metal chains set on 4^{th} level of equipment unit may be designed with hooks and magnets in the middle of conveyer belt or chain to hang pairs (set of) slipper for detachable use hands free, this may include foot press paddle to convey conveyer function moving forward in a loop of equipment unit.

7. That the claim is, hygienic footwear equipment rack unit's conveyer belts and metal chains sets may be vertically escalated to role as rollercoaster loop behind shoe compartment and shoe rack of the equipment unit to accommodate or higher numbers of pairs sets available on conveyer of newly designed slippers, for high volume use or high foot traffic area.

8. That the claim is, hygienic footwear equipment rack units shoe compartment door may be single unit or set of two swingable glass, fiberglass or punched holes metal and or wood with attach hinges, and this to clear view or visible see through doors holes for sterilizing UV process, where ultra violet lamps function in close compartment within the equipment unit lamps turned on with ac/dc power through wall slot plug or battery terminal for sterilizing.

9. That the claim is, hygienic footwear equipment rack units bottom tray drawers on of 5^{th} and 6^{th} level for sanitizing and wiping of footwear sole from the bottom may be a detachable tray manually or automated functional attach ac/dc power electric motor for hands free user's access to WI drawl both drawer open close eject function from the front by the equipment's remote control device, power switch and or manually.

10. That the claim is, hygienic footwear equipment rack units hand held remote device may be set to use equipment's, all electric components function hands free by remote device for each level of equipment unit functions.

11. That the claim is, hygienic footwear equipment rack unit or units may use in hospitals and clinics by all to maintain cleanliness and hygiene of the indoor environment for health and safety reasons prior entering designated care units.

12. That the claim is, hygienic footwear equipment rack unit or units may be provided or used in religious institutions, where hygienic sanitize or sterilized newly designed slippers for use in institutions by visitors' and members.

13. That the claim is, hygienic footwear equipment rack unit or units may be used generally by domestic user, family members or visiting guest before entering the home as traditional conventional practice for health and safety reasons, domestic hygiene and environmental separation (indoor and outdoor) with user friendly features of handling all kind of footwear within equipment unit.

14. That the claim is, hygienic footwear equipment rack units or unit may be provided to or used by general offices or outlet, employees and or customers, where indoor cleanliness and hygiene of premises or public health and safety at higher risk or priority.

15. That the claim is, hygienic footwear equipment rack unit or units may be used in community centers by general public during functions.

16. That the claim is, hygienic footwear equipment rack unit or units may be provided or used by hospitality group hotels, motels class for their customers or and member staff for hotel rooms or some parts of hotel area of relaxation, yoga, health and fitness centers or human safety, as priority of hygiene to maintain.

17. That the claim is, hygienic footwear equipment rack unit or units may provide or used for close shoes treatment for drying of sweat, odor or wetness due to damp shoe after cleaning.

18. The process in sterilizing for bacterial, fungal, or virus causing germs and spreadable infectious disease with hot air blower and UV rays protection from inside the close shoe by electric UV lamp fitted with hot air blower hose (pipe tunnel tube) when connected to shoe inside for up to certain time frame, timer switch function.

18. That the claim is, newly designed slippers may be used with extendable telescopic two flat bars sided within another bar or tubes (areal type) expandable in length size from the middle center between heals and toe for adjustment of slippers pair size from 1 to 4, 5 to 9 and 10 to 16 for footwear's to slip in wear the front flap of newly designed slipper with the foot size adjustment of the footwear or the foot.

19. That the claim is, hygienic footwear equipment rack unit's newly designed slippers may be in three to four layered bonded set pair or one piece hot molded dye material with telescopic flat bars or tubes of metal or plastic inserted in the middle section of the slippers part from heals to toe's, end to end for length size adjustments.

20. That the claim is, hygienic footwear equipment rack unit's newly designed slippers on conveyer loop belts and or metal chains. The middle center of belts and metal chains may be fitted with hanger hooks and magnet pieces or strips on roller at marginal distance of slippers pairs in a row. This to hang or detach slippers from the conveyer chain.

21. That the claim is, the hygienic footwear equipment rack unit and its components units may be comprise and constructed of plastic or metal includes conveyer chains, baring's freewheel, hot air blower parts and casing, shoe compartment and shoe rack.
